(19) United States
(12) Patent Application Publication
(10) Pub. No.: US 2023/0201406 A1
(43) Pub. Date: Jun. 29, 2023

(54) LASER SYSTEMS, METHODS AND DEVICES OF PROCESSING AND SANITIZING AIR FLOW AND SURFACES

(71) Applicant: Nuburu, Inc., Centennial, CO (US)

(72) Inventor: Mark Zediker, Castle Rock, CO (US)

(73) Assignee: Nuburu, Inc., Centennial, CO (US)

(21) Appl. No.: 17/919,117

(22) PCT Filed: Apr. 14, 2021
(86) PCT No.: PCT/US21/27344

§ 371 Date: Oct. 14, 2022

Related U.S. Application Data
(60) Provisional application No. 63/009,769, filed on Apr. 14, 2020.

(51) Int. Cl. A61L 9/18

(52) U.S. Cl. CPC A61L 9/18; B64D 13/06

(57) ABSTRACT

There are provided methods and systems of destroying pathogens, such as virus, flu viruses and bacteria in spaces and surfaces, including confided spaces and surface, including air circulation systems, HVAC systems, and air handling systems in locations such as in offices, hospitals, airplanes, restaurants, cruise ships, hotels, using lasers, coherent light, electromagnetic energy, high intensity photons, including blue lasers and green lasers.

This application claims the benefit of priority to, and under 35 U.S.C. § 119(e)(1) the benefit of the filing date of, U.S. provisional application Ser. No. 63/009,769 filed Apr. 14, 2020, the entire disclosure of which is incorporated herein by reference.

### BACKGROUND OF THE INVENTION

### Field of the Invention

The present inventions relate to laser systems and methods for treating and removing pathogens, and other harmful materials, from surfaces, contaminated buildings, structures and vessels, and air flows. In embodiments laser systems treat air flow in vehicles, aircraft, builds, etc., using laser beams having a wavelength from about 10 nm to about 700 nm, the treating laser beams remove, destroy, or otherwise render the target, e.g., pathogens, safe and no longer dangerous to animals, including humans. In preferred embodiments the laser beam has a blue wavelength.

Infrared red (IR) based (e.g., having wavelengths greater than 700 nm, and in particular wavelengths greater than 1,000 nm).

As used herein, unless expressly stated otherwise, “UV”, “ultra violet”, “UV spectrum”, and “UV portion of the spectrum” and similar terms, should be given their broadest meaning, and would include light in the wavelengths of from about 10 nm to about 400 nm, and from 10 nm to 400 nm, and all wavelengths coming within these ranges.

As used herein, unless expressly stated otherwise, the terms “laser diode”, “diode emitter”, “laser diode bar”, “laser diode chip”, and “emitter” and similar such terms are to be given their broadest meaning. Generally, the laser diode is a semiconductor device that emits a laser beam, such devices are commonly referred to as edge emitting laser diodes because the laser light is emitted from the edge of the substrate. Typically, diode Lasers with a single emission region (Emitter) are typically called laser diode chips, while a linear array of emitters is called laser diode bars. The area emitting the laser beam is referred to as the “facet.”

As used herein, unless expressly stated otherwise, the terms “high power”, lasers and laser beams and similar such terms, mean and include laser beams, and systems that provide or propagate laser beams that have at least 100 Watts (W) of power as well as greater powers, for example from 100 Watts to 10 kW (kilowatts), from about 100 W to about 1 kW, from about 500 W to about 5 kW, from about 10 kw to about 40 kW, from about 5 kW to about 100 kW, and all powers within these ranges, as well as higher powers.

As used herein, unless expressly states otherwise, the term “sanitizing laser intensity”, “sanitizing beam”, “sanitizing” and similar such terms, when used regarding a laser beam, is the intensity of the laser beam in power/cross sectional area of the laser beam that has the ability to destroy, ablate, inactivate, kill, render inert, or render harmless, a pathogen or harmful material. In embodiments sanitizing laser beam intensities include intensities of 100 W (watts)/cm² to 10 kW(kilowatts)/cm², from about 500 W/cm² to about 5 kW/cm², from about 2 kW/cm² to about 1,500 W/cm², about 100 W/cm² and greater, about 500 W/cm² and greater, about 800 W/cm² and greater, about 1,000 W/cm² and greater, about 1,500 W/cm² and greater intensities. These intensities can be provided by focused and shaped high power laser beams, in the blue, green, UV and visible wavelengths.

As used herein, unless expressly stated otherwise, the terms “blue laser beams”, “blue lasers” and “blue” should be given their broadest meaning, and in general refer to systems that provide laser beams, laser beams, laser sources, e.g., lasers and diodes lasers, that provide, e.g., propagate, a laser beam, or light having a wavelength from about 400 nm to about 495 nm, from 400 nm to 495 nm, and all wavelengths within these ranges. Typical blue lasers have wavelengths in the range of about 405-495 nm. Blue lasers include wavelengths of 450 nm, of about 450 nm, of 460 nm, of about 470 nm. Blue lasers can have bandwidths of from about 10 pm (picometer) to about 10 nm, about 5 nm, about 10 nm and about 20 nm, as well as greater and smaller values.

As used herein, unless expressly stated otherwise, the terms “green laser beams”, “green lasers” and “green” should be given their broadest meaning, and in general refer to systems that provide laser beams, laser beams, laser sources, e.g., lasers and diodes lasers, that provide, e.g., propagate, a laser beam, or light having a wavelength from about 500 nm to about 575 nm. Green lasers include wavelengths of 515 nm, of about 515 nm, of 532 nm, about 532 nm, of 550 nm, and of about 550 nm. Green lasers can have bandwidths of from about 10 μm to 10 nm, about 5 nm, about 10 nm and about 20 nm, as well as greater and smaller values.

As used herein, unless expressly stated otherwise, the terms “high reliability”, “highly reliable”, lasers and laser systems and similar terms, mean and include lasers which have a lifetime of at least 10,000 hours or greater, about 20,000 hrs, about 50,000 hours, about 100,000 hours, from about 10 hours to about 100,000 hours, from 10,000 to 20,000 hours, from 10,000 hours to 50,000 hours, from 20,000 hours to about 40,000 hours, from about 30,000 hours to about 100,000 hours and all values within these ranges.

As used herein, unless expressly stated otherwise, the terms “lifetime”, “system lifetime, and “extended lifetime” and similar such terms, are defined as the time during which the output power, other properties, and both of the laser stay at or near a percentage of its nominal value (“nominal value” is the greater of (i) the laser's rated power, other properties, and both, as defined or calculated by the manufacturer, or (ii) the initial power, other properties, and both, of the laser upon first use, after all calibrations and adjustments have been performed). Thus, for example, an “80% laser lifetime” is defined as the total operating time when the laser power, other properties, and both remains at 80% of the nominal value. For example, a “50% laser lifetime” is defined as the total operating time when the laser power, other properties, and both remains at 50% of the nominal value. As used herein, unless specified otherwise or otherwise clear from the context, the term “lifetime” as used herein is referring to an “80% life time”.

As used herein, unless expressly stated otherwise the term “pathogen” should be given its broadest possible means in would include any organism that can cause a disease or condition in animals (including humans, pets and livestock) or plants. Pathogens would include, for example, viruses, bacteria, fungi, molds, and parasites. Pathogens would include, for example, among others anthrax, influenza viruses, corona viruses, COVID-19, SARS-CoV-2, Ebola, HIV, SARS, H1N1 and MRSA.

As used herein, unless expressly stated otherwise the term “harmful material” should be given its broadest possible meaning and would include any material that can cause a disease or condition in animals (including humans, pets and livestock) or plants. Harmful materials would include, for example, particles, metal particle, pathogens, pathogenic materials, spores, biohazards, poisons, toxins and allergens.

As used herein, unless expressly stated otherwise, the term “air handling system”, “air control systems” and “air systems” should be given their broadest possible meaning and would include recirculating air systems, HVAC (heating ventilation air condition) systems, auxiliary handling units, blower systems, heating systems, cooling systems, heating and cooling systems, heating and air conditioning systems for homes and offices, heating and air conditioning systems for airplanes, heating and air conditioning systems for buses, heating and air conditioning systems for cars, heating and air conditioning systems for buildings and heating and air conditioning systems for structures.

As used herein, unless expressly stated otherwise, the terms “recirculating air system”, “recirculation” and similar such terms, means any air handling system where less than 100% of the air moved by the system comes from an exterior source, e.g., fresh air. Thus, recirculating air systems would include systems that recycle (e.g., use air from within the enclosed environment or structure) at least about 10% of the total volume of air in the environment or structure, at least about 50% of the total volume of air in the environment or structure, at least about 75% of the total volume of air in the environment or structure, from about 25% to about 70% of the total volume of air in the environment or structure, and from about 35% to about 50% of the total volume of air in the environment or structure. Closed air systems would include systems that once closed or started up, during operation bring into the environment or structure 5% or less, 10% or less, 25% or less, 50% or less, 75% or less, of their air from external sources, e.g., fresh air.

Generally, the term “about” and the symbol “—” as used herein, unless specified otherwise, is meant to encompass a variance or range of ±10%, the experimental or instrument error associated with obtaining the stated value, and preferably the larger of these.

As used herein, unless expressly stated otherwise terms such as “at least”, “greater than”, also mean “not less than”, i.e., such terms exclude lower values unless expressly stated otherwise.

As used herein, unless stated otherwise, room temperature is 25° C. And, standard temperature and pressure is 25° C. and 1 atmosphere. Unless expressly stated otherwise all tests, test results, physical properties, and values that are temperature dependent, pressure dependent, or both, are provided at standard temperature and pressure.

As used herein, unless specified otherwise, the recitation of ranges of values, a range, from about “x” to about “y”, and similar such terms and quantifications, serve as merely shorthand methods of referring individually to separate values within the range. Thus, they include each item, feature, value, amount or quantity falling within that range. As used herein, unless specified otherwise, each and all individual points within a range are incorporated into this specification, and are a part of this specification, as if they were individually recited herein.

As used herein, unless expressly states otherwise, Class I, Class II, Class III, and subsets of these Classes, refer to systems will meet the requirements of 21 C.F.R. § 1040.10 (Revised as of Apr. 1, 2012), the entire disclosure of which is incorporated herein by reference, portions of which are also set forth in this specification.

As used in this specification a “Class I product” is equipment that will not permit access during the operation of the laser to levels of laser energy in excess of the emission limits set forth in Table I. Thus, preferably personnel operating, and in the area of operation, of the equipment will receive no more than, and preferably less than, the following exposures in Table I during operation of the laser equipment.

TABLE I
CLASS I ACCESSIBLE EMISSION LIMITS FOR LASER RADIATION

| Wavelength | Emission duration | Class I-Accessible emission limits | | |
|---|---|---|---|---|
| (nanometers) | (seconds) | (value) | (unit) | (quantity)** |
| ≥180 but ≤400 | ≤3.0 × 10⁴ | 2.4 × 10⁻⁵k₁k₂ | Joules(J)* | radiant energy |
| | >3.0 × 10⁴ | 8.0 × 10⁻¹⁰k₁k₂ | Watts(w)* | radiant power |
| >400 but | >1.0 × 10⁻⁹ to 2.0 × 10⁻⁵ | 2.0 × 10⁻⁷k₁k₂ | J | radiant energy |
| ≤1400 | >2.0 × 10⁻⁵ to 1.0 × 10¹ | 7.0 × 10⁻⁴k₁k₂t^{3/4} | J | radiant energy |
| | >1.0 × 10¹ to 1.0 × 10⁴ | 3.9 × 10⁻³k₁k₂ | J | radiant power |
| | 1.0 × 10⁴ | 3.9 × 10⁻⁷k₁k₂ | W | radiant energy |
| | and also (See paragraph (d)(4) of this section). | | | |
| | >1.0 × 10⁻⁹ to 1.0 × 10¹ | 10k₁k₂t^{1/3} | Jcm⁻²sr⁻¹ | integrated radiance |
| | >1.0 × 10¹ to 1.0 × 10⁴ | 20k₁k₂ | Jcm⁻²sr⁻¹ | integrated radiance |
| | >1.0 × 10⁴ | 2.0 × 10⁻³k₁k₂ | Wcm⁻²sr⁻¹ | radiance |
| >1400 but | >1.0 × 10⁻⁹ to 1.0 × 10⁻⁷ | 7.9 × 10⁻⁵k₁k₂ | J | radiant energy |
| ≤2500 | >1.0 × 10⁻⁷ to 1.0 × 10¹ | 4.4 × 10⁻³k₁k₂t^{1/4} | J | radiant energy |
| | >1.0 × 10¹ | 7.9 × 10⁻⁴k₁k₂ | W | radiant power |
| >2500 but | >1.0 × 10−⁹ to 1.0 × 10−⁷ | 1.0 × 10⁻²k₁k₂ | Jcm⁻² | radiant exposure |
| ≤1.0 × 106 | >1.0 × 10−⁷to 1.0 × 10¹ | 5.6 × 10 | Jcm⁻² | radiant exposure |
| | >1.0 × 10¹ | 1.0 × 10⁻¹k₁k₂t | Jcm⁻² | radiant exposure |

*Class I accessible emission limits for wavelengths equal to or greater than 180 nm but less than or equal to 400 nm shall not exceed the Class I asscessible emission limits for the wavelengths greater than 1400 nm but less than or equal to 1.0 × 10⁶ nm with a k₁ and k₂ of 1.0 for comparable sampling intervals.
**Measurement parameters and test conditions shall be in accordance with paragraphs (d)(1), (2), (3), and (4), and (e) of this section.

As used in this specification a “Class IIa product” is equipment that will not permit access during the operation of the laser to levels of visible laser energy in excess of the emission limits set forth in Table II-A; but permit levels in excess of those provided in Table I.

TABLE II-A
CLASS IIa ACCESSIBLE EMISSION LIMITS FOR LASER RADIATION
CLASS IIa ACCESSIBLE EMISSION LIMITS ARE IDENTICAL TO CLASS I ACCESSIBLE
EMISSION LIMITS EXCEPT WITHIN THE FOLLOWING RANGE OF WAVELENGTHS AND
EMISSION DURATIONS:

| Wavelength | Emission duration | Class IIa-Accessible emission limits | | |
|---|---|---|---|---|
| (nanometers) | (seconds) | (value) | (unit) | (quantity)* |
| >400 but ≤710 | >1.0 × 10³ | 3.9 × 10⁻⁶ | W | radiant power |

*Measurement parameters and test conditions shall be in accordance with paragraphs (d)(1), (2), (3), and (4), and (e) of this section.

As used in this specification a “Class II product” is equipment that will not permit access during the operation of the laser to levels of laser energy in excess of the emission limits set forth in Table II; but permit levels in excess of those provided in Table II-A.

TABLE II
CLASS II ACCESSIBLE EMISSION LIMITS FOR LASER RADIATION
CLASS II ACCESSIBLE EMISSION LIMITS ARE IDENTICAL TO CLASS I ACCESSIBLE EMISSION LIMITS
EXCEPT WITHIN THE FOLLOWING RANGE OF WAVELENGTHS AND EMISSION DURATIONS:

| Wavelength | Emission duration | Class II-Accessible emission limits | | |
|---|---|---|---|---|
| (nanometers) | (seconds) | (value) | (unit) | (quantity)* |
| >400 but ≤710 | >2.5 × 10⁻¹ | 1.0 × 10⁻³ | W | radiant power |

*Measurement parameters and test conditions shall be in accordance with paragraphs (d)(1), (2), (3), and (4), and (e) of this section.

As used in this specification a “Class IIIa product” is equipment that will not permit access during the operation of the laser to levels of laser energy in excess of the emission limits set forth in Table III-A; but permit levels in excess of those provided in Table II.

TABLE III-A
CLASS IIIa ACCESSIBLE EMISSION LIMITS FOR LASER RADIATION
CLASS IIIa ACCESSIBLE EMISSION LIMITS ARE IDENTICAL TO CLASS I ACCESSIBLE
EMISSION LIMITS EXCEPT WITHIN THE FOLLOWING RANGE OF
WAVELENGTHS AND EMISSION DURATIONS:

| Wavelength | Emission duration | Class IIIa-Accessible emission limits | | |
|---|---|---|---|---|
| (nanometers) | (seconds) | (value) | (unit) | (quantity)* |
| >400 but ≤710 | >3.8 × 10⁻⁴ | 5.0 × 10⁻³ | W | radiant power |

*Measurement parameters and test conditions shall be in accordance with paragraphs (d)(1), (2), (3), and (4), and (e) of this section.

As used in this specification a “Class IIIb product” is equipment that will not permit access during the operation of the laser to levels of laser energy in excess of the emission limits set forth in Table III-B; but permit levels in excess of those provided in Table III-A.

TABLE III-B
CLASS IIIb ACCESSIBLE EMISSION LIMITS FOR LASER RADIATION

| Wavelength | Emission duration | Class IIIb-Accessible emission limits | | |
|---|---|---|---|---|
| (nanometers ) | (seconds) | (value) | (unit) | (quantity)* |
| ≥180 but ≤400 | <2.5 × 10⁻¹ | 3.8 × 10⁻⁴k₁k₂ | J | radiant energy |
| out | >2.5 × 10⁻¹ | 1.5 × 10⁻³k₁k₂ | W | radiant power |
| >400 but | >1.0 × 10⁻⁹ to | 10k₁k₂t^{1/3} | Jcm⁻² | radiant exposure |
| ≤1400 | 2.5 × 10⁻¹ | to a maximum value | Jcm⁻² | radiant exposure |
| | | of 10 | W | radiiant power |
| | | 5.0 × 10⁻¹ | | |
| >1400 but | >1.0 × 10⁻⁹ to | 10 | Jcm⁻² | radiant exposure |
| but ≤1.0 × 106 | >1.0 × 10¹ | 5.0 × 10⁻¹ | W | radiant power |

The values for the wavelength dependent correction factors “k1” and “k2” for Tables I, IIA, II, IIIA, IIIB are provided in Table IV.

TABLE IV
VALUES OF WAVELENGTH DEPENDENT CORRECTION FACTORS k₁ AND k₂

| Wavelength | | | | |
|---|---|---|---|---|
| (nanometers) | k₁ | k₂ | | |
| 180 to 302.4 | 1.0 | 1.0 | | |
| > 302.4 to 315 | ${10}^{[ \frac{λ - 302.4}{5} ]}$ | 1.0 | | |
| > 315 to 400 | 330.0 | 1.0 | | |
| > 400 to 700 | 1.0 | 1.0 | | |
| > 700 to 800 | ${10}^{[ \frac{λ - 700}{515} ]}$ | $\begin{matrix}if : t \leq \frac{10100}{λ - 699} \\ then : {k}_{2} = 1.\end{matrix}$ | $\begin{matrix}if : \frac{10100}{λ - 699} < t \leq {10}^{4} \\ then : {k}_{2} = \frac{t ( λ - 699 )}{10100}\end{matrix}$ | $\begin{matrix}if : t > {10}^{4} \\ then : {k}_{2} = \frac{λ - 699}{1.01}\end{matrix}$ |
| > 800 to 1060 | ${10}^{[ \frac{λ - 700}{515} ]}$ | if: t ≤ 100 then: k₂ = 1.0 | $\begin{matrix}if : 100 < t \leq {10}^{4} \\ then : {k}_{2} = \frac{t}{100}\end{matrix}$ | if: t > 10⁴ then: k₂ = 100 |

| | | |
|---|---|---|
| > 1060 to 1400 | 5.0 | |
| > 1400 to 1535 | 1.0 | 1.0 |
| > 1535 to 1545 | t ≤ 10⁻⁷ | 1.0 |
| | k₁ = 100.0 | |
| | t > 10⁻⁷ | |
| | k₁ = 1.0 | |
| > 1545 to | 1.0 | 1.0 |
| 1.0 × 10⁶ | | |

Note:
The variables in the expressions are the magnitudes of the sampling interval (t) , in units of seconds, and the wavelength (λ), in units of nanometers.

The measurement parameters and test conditions for Tables I, IIA, II, IIIA, and IIIB, which are referred to by paragraph numbers of “this section,” are as follows, and are provided with their respective paragraph numbers “b” and “e” as they appear in 21 C.F.R. § 1040.10 (Revised as of Apr. 1, 2012):

(b)(1)Beam of a single wavelength. Laser or collateral radiation of a single wavelength exceeds the accessible emission limits of a class if its accessible emission level is greater than the accessible emission limit of that class within any of the ranges of emission duration specified in tables I, II-A, II, III-A, and III-B.

(b)(2)Beam of multiple wavelengths in same range. Laser or collateral radiation having two or more wavelengths within any one of the wavelength ranges specified in tables I, II-A, II, III-A, and III-B exceeds the accessible emission limits of a class if the sum of the ratios of the accessible emission level to the corresponding accessible emission limit at each such wavelength is greater than unity for that combination of emission duration and wavelength distribution which results in the maximum sum.

(b)(3)Beam with multiple wavelengths in different ranges.” Laser or collateral radiation having wavelengths within two or more of the wavelength ranges specified in tables I, II-A, II, III-A, and III-B exceeds the accessible emission limits of a class if it exceeds the applicable limits within any one of those wavelength ranges.

(b)(4)Class I dual limits. Laser or collateral radiation in the wavelength range of greater than 400 nm but less than or equal to 1.400 nm exceeds the accessible emission limits of Class I if it exceeds both: (i) The Class I accessible emission limits for radiant energy within any range of emission duration specified in table I, and (ii) The Class I accessible emission limits for integrated radiance within any range of emission duration specified in table I.

(e) (1)Tests for certification. Tests shall account for all errors and statistical uncertainties in the measurement process. Because compliance with the standard is required for the useful life of a product such tests shall also account for increases in emission and degradation in radiation safety with age.

(e)(2)Test conditions. Tests for compliance with each of the applicable requirements of paragraph (e) shall be made during operation, maintenance, or service as appropriate: (i) Under those conditions and procedures which maximize the accessible emission levels, including start-up, stabilized emission, and shut-down of the laser product; and (ii) With all controls and adjustments listed in the operation, maintenance, and service instructions adjusted in combination to result in the maximum accessible emission level of radiation; and (iii) At points in space to which human access is possible in the product configuration which is necessary to determine compliance with each requirement, e.g., if operation may require removal of portions of the protective housing and defeat of safety interlocks, measurements shall be made at points accessible in that product configuration; and (iv) With the measuring instrument detector so positioned and so oriented with respect to the laser product as to result in the maximum detection of radiation by the instrument; and (v) For a laser product other than a laser system, with the laser coupled to that type of laser energy source which is specified as compatible by the laser product manufacturer and which produces the maximum emission level of accessible radiation from that product.

(e)(3)Measurement parameters. Accessible emission levels of laser and collateral radiation shall be based upon the following measurements as appropriate, or their equivalent: (i) For laser products intended to be used in a locale where the emitted laser radiation is unlikely to be viewed with optical instruments, the radiant power (W) or radiant energy (J) detectable through a circular aperture stop having a diameter of 7 millimeters and within a circular solid angle of acceptance of 10⁻³ steradian with collimating optics of 5 diopters or less. For scanned laser radiation, the direction of the solid angle of acceptance shall change as needed to maximize detectable radiation, with an angular speed of up to 5 radians/second. A 50 millimeter diameter aperture stop with the same collimating optics and acceptance angle stated above shall be used for all other laser products. (ii) The irradiance (W/cm²) or radiant exposure (J/cm²) equivalent to the radiant power (W) or radiant energy (J) detectable through a circular aperture stop having a diameter of 7 millimeters and, for irradiance, within a circular solid angle of acceptance of 10⁻³ steradian with collimating optics of 5 diopters or less, divided by the area of the aperture stop (cm²). (iii) The radiance (W/cm² steradian) or integrated radiance (J/cm² steradian) equivalent to the radiant power (W) or radiant energy (J) detectable through a circular aperture stop having a diameter of 7 millimeters and within a circular solid angle of acceptance of 10⁻⁵ steradian with collimating optics of 5 diopters or less, divided by that solid angle (sr) and by the area of the aperture stop (cm²).

Harmful materials, such as pathogens, anthrax, coronavirus, 2019-nCoV, SARS (Sever Acute Respiratory Syndrome), Methicillin-resistant Staphylococcus aureus (MRSA), flu virus, and other pathogens, and in particular air borne pathogens, present significant risks to humans, pets, livestock and plants. This risk is heightened and of significant concern when groups are located in enclosed, or partially enclosed, spaces having air handling systems. These risks are present, for example, in theaters, airplanes, busses, airports, hotels, hospitals, churches, mosques, temples, synagogues, office buildings, jails, homes, automobiles, shopping malls, stores, arenas, schools, green houses, growing houses, poultry houses, chicken farms, horse barns, zoos and kennels.

Enclosed spaces with air handling system, such as air conditioning, whether heating or cooling, puts people at risk to contamination and harm from, pathogens, such as viruses and bacteria, allergens such as mold and fungus and spores such as anthrax, to name a few. These risks can be both from external air sources (e.g. allergens, pollutants), and cross contamination from others within in the enclosed space. The risk of cross contamination is heighted, and increased, by the use of recirculating air systems, with this risk increasing as greater amounts (e.g., volumes or percentages of total volume) of air is recycled and reused.

These risks of enclosed environments have become increasingly important, and of greater significance, with the global economy, free movement of peoples and livestock, and with larger and larger structures, and longer and longer times of confinement. By way of illustration a Boing an Airbus A380 can hold over 500 people for flights that can last 15 hours or longer. A Boeing 777 can hold 317-396 people for flights that can last over 15 hours. Large multi-use (residential and commercial) high-rises buildings can house thousands of people for extend periods of time, days, weeks months and years. Cruise ships are becoming larger and larger, holding more and more passenger (e.g., 5,000 and more passengers with 2,000 or more crew) for extended voyages (e.g., several days, weeks, and months).

This Background of the Invention section is intended to introduce various aspects of the art, which may be associated with embodiments of the present inventions. Thus, the foregoing discussion in this section provides a framework for better understanding the present inventions, and is not to be viewed as an admission of prior art.

### SUMMARY

There is a continuing and increasing need to remove harmful materials, as well as, annoying materials such as odors, from air handing systems and enclosed spaces. This need is particularly significant and long felt for environments that use recirculating air systems. This need has become increasingly important, and unmeet, with larger and larger structures, and longer and longer times of confinement. The present inventions solve these needs, among other things, by providing the improvements, articles of manufacture, devices and processes taught, and disclosed herein.

There is provided a laser system having a sanitizing laser beam to mitigating harmful materials in a gas stream, the laser system comprising: a laser for generating a sanitizing laser beam along a laser beam path; a housing, the housing defining an optically active area; wherein, the optically active area is on the laser beam path and thereby in optical communication with the laser; the laser beam path extended into the optically active area; and thereby defining a portion of the laser beam path as an optically active laser beam path; whereby the optically active laser beam path is located within the optically active area; and, the optically active area configured to have a gas stream flow through the optically active area; whereby during operation the gas stream flows through the sanitizing laser beam on the optically active laser beam path.

Moreover, there is provided a laser system having a sanitizing laser beam to mitigating harmful materials in a gas stream, the laser system comprising: a laser for generating a laser beam along a laser beam path; an optically active area, comprising a plurality of laser beam directing devices to define an optically active laser beam path; the optically active laser beam path defining an illumination zone; and, the optically active laser beam path in optical communication with the laser beam path, and thereby forming a part of the laser beam path; wherein, the system is configured to provide a laser power density in the illumination zone to mitigate a harmful material.

Still further, there is provided a laser system having a sanitizing laser illumination zone for mitigating harmful materials, the laser system comprising: a laser for generating a laser beam along a laser beam path; an optically active area, defining an illumination zone; wherein, at least a portion of the laser beam path is within the optically active area; and, the laser beam path extended into the optically active area; and thereby defining a portion of the laser beam path as an optically active laser beam path; whereby the optically active laser beam path is located within the optically active area; and, the system is configured whereby the illumination zone is a sanitizing illumination zone.

In addition, there is provided a method of using a sterilizing laser beam to render a contaminated surface safe for humans and mammals, the method comprising: directing a sanitizing laser beam onto a surface contaminated with a harmful material, wherein the satanizing laser beam strikes the surface for a period of time and at a power density in W/cm2, wherein the time and power density are such that the harmful material is rendered safe, without damaging the surface.

Moreover, there is provided the method of using a laser system to mitigate pathogens from an air flow stream.

Moreover, there is provided the method of using non-ionizing radiation to mitigate pathogens from an air flow stream.

Yet further there are provided These laser systems and methods having one or more of the following features: further having optics for defining the shape of the laser beam; wherein the laser beam in the optically active area comprises a beam cross section shape selected from the group consisting of circular, square, rectangular, and linear; wherein a plurality of beam directing devices are located on the optically active laser beam path; wherein the plurality of beam directing devices comprise a first reflecting device and a second reflecting device, wherein each reflecting device is oriented at an angle to the laser beam path, wherein the angle is other than 90¬∫; wherein the angle is from about 0.5¬∫ to about 15¬∫; wherein the angle for the first reflecting device and the second reflecting device are the same; wherein the angle for the first reflecting device and the second reflecting device are different; wherein the angle for at least one of the reflecting devices is about 1¬∫; wherein one, both or all of the reflecting devices comprises a reflecting device selected from the group consisting of a mirror, a mirror having at least 99% reflectivity for the laser, and a total internal reflection optic; wherein the optically active laser beam path is oriented in a non-resonate configuration; further having a laser beam reversing device located along the laser beam path, whereby the direction of the laser beam is reversed, thereby defining a reverse optically active laser beam path, at least a portion of the reverse optically active laser beam path located within the optically active area; wherein the laser beam reversing device is a mirror orientated at a 90¬∫ angle to the laser beam; further having a beam dump; wherein there is provided a power density of from 500 W/cm2 to 1,000 W/cm2 within the optically active area; wherein there is provided a power density of from 500 W/cm2 to 2,000 W/cm2 within the optically active area; wherein there is provided a power density of at least 500 W/cm2 within the optically active area; wherein there is provided a power density of at least 750 W/cm2 within the optically active area; wherein the optically active area is configured to mitigate the harmful materials by weakening an outer shell of a virus; wherein the optically active area is configured to mitigate the harmful materials by heating a virus, bacteria, or both to a temperature that renders the virus, bacteria or both safe, inactive or dead; wherein the optically active area is configured to mitigate the harmful materials by ablating the harmful material; wherein the harmful material are one or more of a pathogen, pathogenic material, spore, biohazard, poison, toxin, allergen, anthrax, influenza viruses, corona viruses, COVID-19, SARS-CoV-2, Ebola, HIV, SARS, H1N1 and MRSA; wherein a wavelength of the laser beam is selected for optimum absorption by the harmful material; wherein the laser beam has a wavelength from about 380 nm to 1500 nm; wherein the laser beam has a wavelength that is within the blue wavelengths; wherein the laser beam has a wavelength that is within the blue-green wavelengths; wherein the laser beam has a wavelength that is within the UV wavelengths; wherein the laser beam has a wavelength of about 450 nm, about 460 nm, or about 470 nm; wherein the laser beam has a bandwidth of from about 10 pm (picometer) to about 10 nm, about 5 nm, about 10 nm, or about 20 nm; wherein the housing meets the requirement of a Class III, more preferably Class II, (and sub-sets of these Classes) and still more preferably Class I laser system; wherein the laser system is a Class III, more preferably Class II, (and sub-sets of these Classes) and still more preferably Class I; wherein the laser system or method is association with an air handling system; and, wherein the laser system or method is association with a return air stream of an air handling system.

Still further there is provided these laser systems and methods having one or more of the following features: wherein mitigation comprises by weakening an outer shell of a virus; wherein mitigation comprises raising the temperature of a virus, a bacteria or both, to a temperature that in actives, renders inert, or kills the virus, the bacteria or both; wherein mitigation comprises ablating a virus, a bacteria or both; wherein mitigation comprises one or more of destroying, ablating, inactivating, killing, rendering inert, or rendered harmless; wherein the laser comprises a Raman laser pumped by a blue laser diode; wherein the laser comprises a UV laser diode; wherein the laser comprises a visible laser diode; wherein the laser comprises visible laser diode; wherein the laser is fiber coupled; further comprising a particle filter upstream of the laser sanitization system to reduce dust and other debris; further comprising, a particle filter downstream of the laser sanitization system to capture ash and other debris from the laser sanitization system; wherein the downstream particle filter system comprises a blue laser system for gettering carbon; further comprising, a frequency doubled IR laser; wherein the laser beam has an homogenous beam profile; wherein the laser beam has a top hat beam profile; wherein the laser is a visible laser; wherein the laser is a blue lase; wherein the laser is a green laser; wherein the laser beam is delivered from one or more of a robot, a drone, or a remote operated vehicle; wherein the surface is any surface of a material touched by human hands; wherein the material is one or more of clothing or money and other things “touched” by human hands when operated at low power levels; further comprising one or more of a fiber laser, a disk laser or a solid state laser; wherein the system or methods are used in a metal fabrication facility to eliminate airborne particles of metal from the atmosphere; and wherein the metal fabrication facility is an additive manufacturing plant.

### BRIEF DESCRIPTION OF THE DRAWINGS

FIG. 1. is a graph of the distance a particle travels while being heated to a 300 C temperature a 400 CFM air flow with a laser beam having an illumination intensity of 1,000 W/cm² and a wavelength of 450 nm, in accordance with the present inventions.

FIG. 2. is a graph of distance of travel of a particle to vaporize steel particles in an air flow with a laser beam having an illumination intensity of 1 kW/cm² and a wavelength of 450 nm, in accordance with the present inventions.

FIG. 3 is a cross sectional schematic diagram of an embodiment of an air duct laser system in accordance with the present inventions.

FIG. 4 is a schematic diagram of an embodiment of an air handling system in accordance with the present inventions.

FIG. 5 is a schematic diagram of an air handling system in accordance with the present inventions.

FIG. 6 is a schematic diagram of an airplane having an air handling system in accordance with the present inventions.

FIG. 7 is a perspective, partial internal view of an air handling module in accordance with the present inventions.

FIG. 8 is a side view of an Auxiliary Handling Unit (AHU) laser system in accordance with the present inventions.

### DESCRIPTION OF THE PREFERRED EMBODIMENTS

The present inventions relate to laser systems and methods for treating and removing pathogens, and other harmful materials, from surfaces, structures, vessels and air flows.

In general, embodiments of the present laser delivery systems have a source of high power laser beam from 10 nm to about 800 nm, preferably in the blue, blue-green and green wavelengths, and more preferably in the blue wavelengths. These systems have beam shaping and directing assemblies that shape the beam into a particular cross sectional shape and intensity, so that the laser beam is sanitizing. This assembly also directs the laser beam along a laser beam path or paths, that fill an area of space, so as to create an optically active area or zone. In this manner any pathogens or hazardous materials that are in the optically active zone, or pass through the optically active zone at a predetermined rate and thus have a predetermined residence time in the optically active zone, will be rendered safe (e.g., destroyed, ablated, inactivated, killed, rendered inert, or rendered harmless). The systems also can have a residual beam management device, which manages the sanitizing laser beam if the beam path extends out of the optically active area. The residual beam management devices can be a beam dump, an actively cooled beam dump, an optic that scatters the beam such that the beam's power will not damage the internal structure of the system, a polarizing reflector that reflects the beam back along its path and into the optical active zone, a beam path of such length that at the end the path the beam is attenuated for all practical purposes, (e.g., it is harmless), and other ways to manage the beam that remains after the formation of the optically active zone. The systems may also, and preferably have safety interlocks and shielding. Preferably, the laser systems and equipment, and air handling systems will meet the requirements of 21 C.F.R. § 1040.10 (Revised as of Apr. 1, 2012), the entire disclosure of which is incorporated herein by reference, to be considered Class III, more preferably Class II, (and sub-sets of these Classes) and still more preferably Class I.

A single laser system can have one or more optically active areas, and these areas can be arranged in any fashion. For example, they can be arranged as a series of parallel planes each of which entirely fills the flow path of the air in an air conduit (e.g., air duct). The optically active zones can be arranged as a series of vertical, horizontal, angular, planes, intersecting, not intersection and both. In this manner when the multiple optically active zones are viewed collectively with the air flow in the laser system, all air passing through the laser system will pass through an optically active zone, and in a manner that sanitizes the air, upon exiting the Laser system. The optical active area can be created by delivering the laser beam in a rapidly scanned pattern. The cross sectional shape of a sanitizing laser beam can be circular, oval, rectangular, square, linear (e.g., ribbon line, having a length that is 10×, 20× or more its width), or any other shape.

While blue laser wavelength are particularly preferred, the use of laser modules having different wavelengths, may be used. Further, if there is a particular harmful material that is being addressed and mitigated, that has a high absorptivity in a particular wavelength, that wavelength could be used to have an optical active zone optimized for that particular material.

One, two, three, four, five, ten, tens, and hundreds of the present laser systems can be used for an air handling system. The laser systems can be arranged to have provide optically active areas so all air moving through the system, and in particular all recycled air, is sanitized by passing through one or more optically active zones. The laser systems can be modules that are added to the air handling conduits of an air handling system, the can be built into the air handling system, they can be part of a heating unit, the can be part of a cooling unit, the can be stand alone unit that receives and processes air from a room or other air handling system, they can be integral with the air handing system, and well as combinations and variation of such uses with air handling systems.

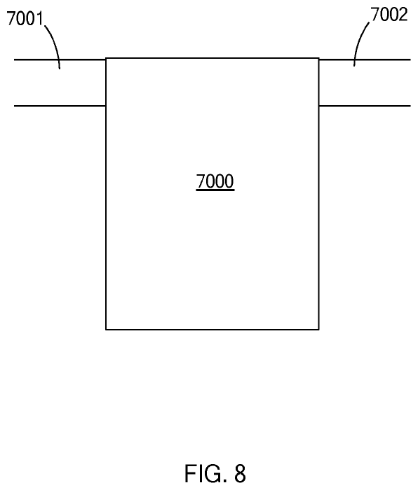

In an embodiment they can be a stand alone modular unit that is used sanitize air flow that is being vented from a contaminated a building, structure, or vessel to as part of a mitigation to contamination. For example, if a building is contaminated with anthrax the building atmosphere can be vented to the outside to reduce the level of contamination within the building. This vented air, will be contaminated with the anthrax and the stand alone modular unit, such as the embodiment of FIG. 8 can be used to remove the anthrax from the vented air.

Embodiments of the present systems uses a blue laser system to render safe, e.g., (e.g., destroyed, ablated, inactivated, killed, rendered inert, or rendered harmless) all forms of airborne pathogens through one of three mechanisms without affecting the temperature of the air being processed. It is theorized that the three basic mechanisms that can kill most viruses and bacteria include: 1) illumination with blue light weakens the outer shell of the virus or bacteria, rending it susceptible to other forms of sterilization such as a hydrogen peroxide wash, 2) heating of the virus or bacteria beyond a temperature that it can withstand, and 3) burning the dust particles, virus, bacteria or spore up with sufficient intensity, e.g. ablating them. Additionally, it is theorized that high intensity blue laser beams have the ability to create reactive oxygen species, which will also kill most viruses and bacteria and other pathogens. The first method can destroy many viruses and bacteria without the need for a hydrogen peroxide wash and will require the least intensity of the three methods described in this invention. This method can destroy all of the MRSA in an optically active area on a surface or tool or through an air flow but will require the longest residence time in the optically active area. The second method can kill all viruses and bacteria with a minimal amount of energy input. A virus can be killed by exposure to sufficient intensity to increase the temperature rapidly to beyond 70 C, taking the worse case virus size into consideration, and increasing the intensity to the level that all potential viruses will be raised to a temperature in excess of 100 C will insure a 99.99% or better kill rate. Bacteria on the other hand has to be raised to a temperature in excess of 130 C to insure it is killed and the size of the bacteria as well as the particles it may be attached to are a major consideration when determining the intensity of the optical field necessary to achieve a 99.99% or better kill rate. The third method uses a sufficiently high optical field to ash or incinerate all microscopic particles which pass through it. This method will yield a very high kill rate with a very low probability of anything surviving. Thus, the use of multiple optically active areas of at the levels of method two or three, which serially treat air flow is a preferred embodiment of a system to assure 100% removal of all pathogens, or hazardous materials of concern, passing through the system.

The use of a blue laser system for sterilization of a HVAC air system has several major advantages over the use of a plasma or UltraViolet system. The blue laser can be delivered by an optical fiber so the system can be very compact. The blue laser light does not cause any deterioration of components in the system like a UV light system might. The laser light can be collimated and therefore it can be used to sterilize any arbitrary size duct system. Finally, air at this wavelength (450 nm) is highly transparent so there will be no thermal distortion of the beam in the sterilization chamber, nor will there be any attenuation of the light as it traverses the sterilization chamber. Consequently, the laser light may be collimated, or un-collimated, launched into an optical system that confines the light in such a way that many overlapping paths for the lap are created. These overlapping paths result in a higher intensity at that position in the sterilization chamber than originally launched into the system. The system may also be set up with non-overlapping regions to create longer exposure times for the particles traversing the sterilization chamber.

While blue lasers wavelengths are preferred for these present embodiments of these air handling sanitization systems and methods, blue-green and green laser wavelength should have good results if utilized while IR lasers will have good results for the third method described, but less efficient than the blue wavelength sources.

Laser beams, being light, are non-ionizing radiation. Thus, embodiments of the present systems and methods provide the ability to mitigate harmful materials and pathogens, without the use of ionizing radiation, and ionizers.

The following examples are provided to illustrate various embodiments of the present systems, apparatus, and methods. These examples are for illustrative purposes, may be prophetic, and should not be viewed as, and do not otherwise limit the scope of the present inventions.

### Example 1—Modeling of Particle Temperature in an Air Flow

For the purpose of this calculation, the baseline system will be a 1 ton HVAC unit at 400 CFM, the duct size will be 10″ round (from standard HVAC tables). The laser will be collimated to create 1 kW/cm² power density and the calculations will assume the density of calcium carbonate, the heat capacity of calcium carbonate and the melting temperature of calcium carbonate and that the blue laser light (450 nm) has an absorption coefficient of 50% on the surface of the calcium carbonate.

The absorption cross section for the laser light is assumed to be illuminated from one direction and is simply the product of the cross-sectional area (π*r²) and the absorption coefficient (σ):

Absorbed Power=π*r²*σ (1)

This is a conservative estimate since the laser light will be bouncing in all directions and there can easily be a factor of 2× in the amount of absorbed power when the particle is being illuminated from two sides.

The time to heat up a particle to a certain temperature can be calculated by dividing the energy required to heat up a mass to a given temperature by the absorbed power:

Time=ρ*Cp*DT/Absorbed Power (2)

Here ρ is the density of the particle, Cp is the heat capacity and DT is the change in temperature.

The last factor needed to determine the interaction length required to achieve a give temperature is the flow velocity of the air in the duct. Using standard air duct specifications, an equivalent diameter duct of 10″ results in a flow velocity (V) of 367 cm/sec. This can then be used to calculate how long of an interaction length is needed to achieve a given temperature.

Length=V*Time (3)

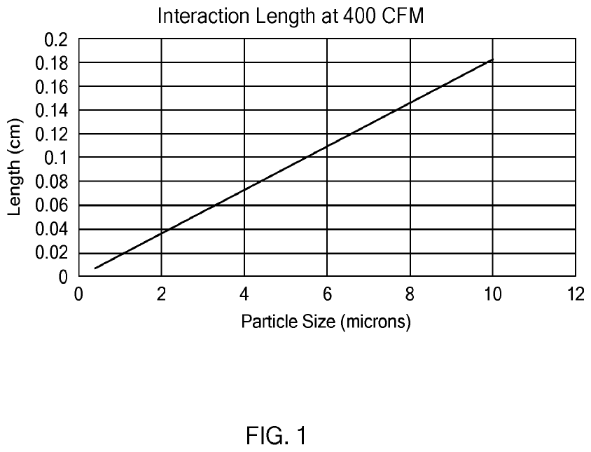

Turning to FIG. 1 there is shown a graph of the interaction lengths needed for a 1 kW/cm² optically active zone (e.g., illumuniated zone) of air duct flowing at 400 CFM as a function of the diameter of the particle. This calculation assumes a rise in temperature of 300 C to ensure that all pathogens are killed in the illuminated zone.

The interaction length may also be lengthened to absorb sufficient energy to vaporize a particle which is the preferred method to eliminate a biohazard such as anthrax. The energy required to vaporize a metallic particle must include two phase changes; melting and vaporization:

Vaporization Energy=Eₜ+H_{f}+Hᵥ (1)

In this equation, Eₜ is the energy required to heat the mass to the desired temperature, H_{f}, is the phase change energy to melt the material, and Hᵥ is the phase change energy to vaporize the material once at temperature.

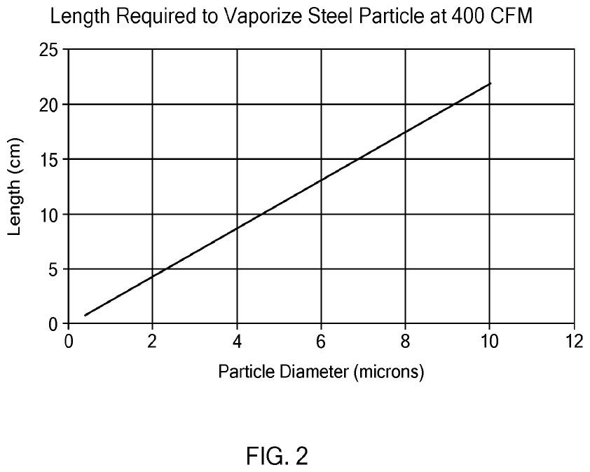

The interaction length calculation is shown in FIG. 2 for the time it would take to vaporize a particle of steel which is a conservative estimate compared to vaporizing an organic such as an anthrax spore. This graph shows that it is possible to superheat a particle until it completely vaporizes in a laminar flow air duct. An organic particle will require substantially less time to ash or vaporize compared to the heavy \steel particles. Shorter interaction lengths can be achieved with higher laser power densities which means a high laser input energy.

### Example 2

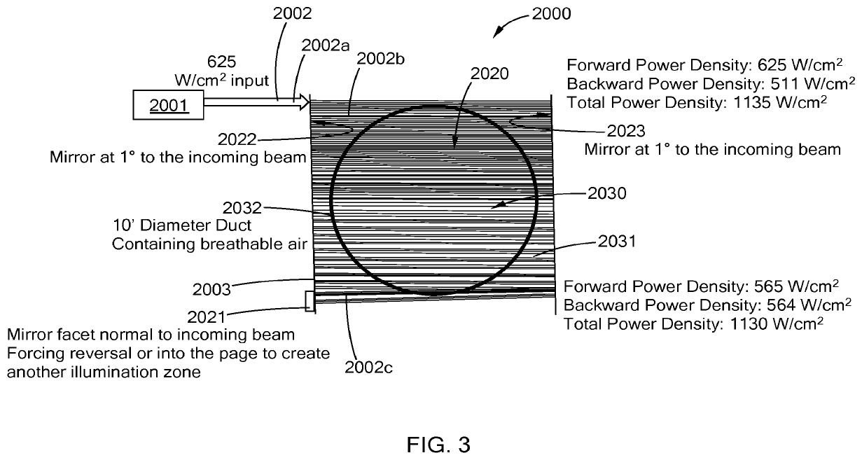

Turning to FIG. 3 there is shown a cross section schematic of an embodiment of a laser beam delivery unit or laser system 2000, having a laser 2001 that provides a laser beam 2002 traveling along an optical path (laser beam path) 2002a, that creates an optically active area or zone 2020, that fills the entire cross sectional area 2030 of a conduit 2032 of an air handling system.

The laser system 2000 has a sanitizing laser beam to mitigating harmful materials in an air flow or gas stream 2031. The laser 2001 generates a laser beam 2002 along a laser beam path 2002a. The housing 2003 contains an optically active area 2020. The optically active area 2020 is in optical communication with the laser 2001. The laser beam path 2002a extends into the optically active area 2020. This portion of the laser beam path 2002a that is within the optically active area 2020 is the optically active laser beam path 2002b. The laser beam 2002 travels from the laser 2001 along the laser beam path 2002a and into the housing 2003 and then back and forth along the optically active laser beam path 2002b until the laser beam reaches the beam return mirror 2021, which is located on the laser beam path 2002a, and in this embodiment also along the optically active laser beam path 2002b. The beam return mirror 2021 directs the laser beam back along the laser beam path 2002a, and in this embodiment also along the optically active laser beam path 2002b. Thus, forming a reverse optically active laser beam path 2002c, which in this embodiment is coincident with the laser beam path 2002a, and the reverse optically active laser beam path 2002c. It is understood that in operation the laser beam travels along these laser beam paths.

In this embodiment portions of the laser beam path 2002a have or incudes the optically active laser beam path 2002b and the reverse optically active laser beam path 2002c.

In this embodiment the housing 2003 is square (it being understood other shapes may be used) and the optically active area 2020 fills the entire area of the housing 2003, as well as the entire cross section 2030 of conduit 2032. In this embodiment a circular conduit 2032 is attached to (in fluid communication with) the square housing 2003. The air flow 2031, from the conduit 2032, fills and travels through the house 2003, and in this manner the entirety of the air flow from conduit 2032 passes through the optically active area 2020, and through the laser beam paths (2002b, 2002c) and thus laser beam 2002, and thus, the air flow is sanitized by the laser beam.

Ray trace analysis in FIG. 3 shows how a plane parallel set of mirrors 2022, 2023 located on the interior opposite walls of the housing 2003, can be orientated in a non-resonate configuration to achieve a near uniform 1100 W/cm2 power density with only two passes through the plane mirror sterilization system.

This unit has a set of high reflectivity mirrors 2022, 2023 with >99% reflectivity, where 99.9% is typical of a narrowband high reflectivity coating. The surfaces of the mirrors are parallel to the flow of air through the optically active zone. This high reflectivity enables the laser beam to be launched at lower than the optimum power density; and as the beam reflects and overlaps itself, the intensity of the beam is readily increased, so that the beam in the optically active zone is a sanitizing beam.

FIG. 3 shows a ray trace of one optical cavity design, here a 625 W/cm² beam is launched into a pair of mirrors that are plane parallel at an angle of 1°. The last pass of the beam will hit a mirror that is tilted at 1° to make it perpendicular to the incoming light. This will cause the beam to retrace itself. In the beam path of this embodiment, there are 50 bounces off of the distal mirror located 30 cm from the input mirror. There are likewise 50 bounces off of the first (input) mirror. The last mirror is tipped normal to the incoming beam to reflect the beam back on itself. After these 200 bounces off of the mirrors, the power density of the beam is still at >511 W/cm². By summing up the power density of the forward going beam with the backward going beam it is possible to achieve a uniform high-power density in a non-resonate cavity all along, i.e., of the entire area of, the sterilization zone as shown in FIG. 3

In this embodiment the depth of the optically active area (what would be viewed as into and from the drawing page, which is a cross section) is on the order of about 1 cm, well in excess of the computations and guidance provided by Example 1 and as shown in FIG. 1, but short of the conditions of FIG. 2 which is the worse case for any airborne component. FIG. 2 was calculated to consider using this in air purification systems in 3D printing plants where fine airborne particles are commonly found. A longer interaction zone can be created by replacing the mirror at the bottom of the non-resonant structure with a mirror pointing into the page (of the figure) and normal to the angle of incidence onto a second mirror that redirects the beam vertically, so the beam retraces vertically up in the picture to fill in the region directly adjacent to the first region. At the top of the structure a mirror that is orthonormal to the incoming beam is added to reflect the beam back down its original path through the two zones. Creating an interaction depth that is now 2 cm deep rather than the original 1 cm. This method can be applied multiple times or parallel zones can be created by using multiple lasers to energize each laser distribution unit, e.g., sanitization cell.

The depth of the optically active area, i.e., the distance that the air flow must travel to pass through (i.e., into and out of) the optically active area can be any distance that provides sufficient residence time for the harmful material in the air flow to be rendered safe by the sanitizing laser beam in the optically active zone. Among other things, the rate of gas flow, the amount of harmful materials, and the power density of the laser beam are factors to considered in determing this distance. By way of example this distance, e.g., the depth, can be from about 0.5 cm to about 5 cm, greater than about 1 cm, greater than about 2 cm, greater than about 3 cm, and longer.

Preferably, the wavelength of the laser beam in this unit of the embodiment of this example is 450 nm.

Ray trace of analysis FIG. 3 shows how a plane parallel set of mirrors can be orientated in a non-resonate configuration to achieve a near uniform 1100 W/cm2 power density with only two passes through the plane mirror sterilization system.

### Example 3

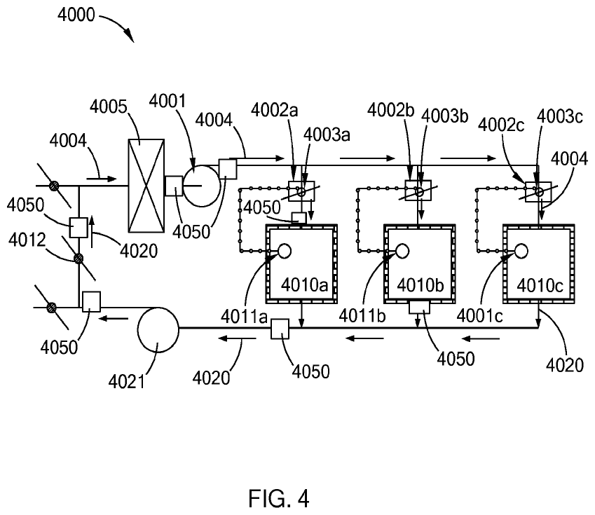

Turning to FIG. 4 there is shown a schematic of an embodiment of a laser air handling system 4000 having one or more laser delivery units 4050. The laser delivery units can be of the type of Example 4. Although not shown in the figure, a laser delivery unit can be positioned to processes incoming (fresh) air. In this embodiment each laser unit has its own laser beam source. The laser delivery units provide one or more sanitizing optical active zones.

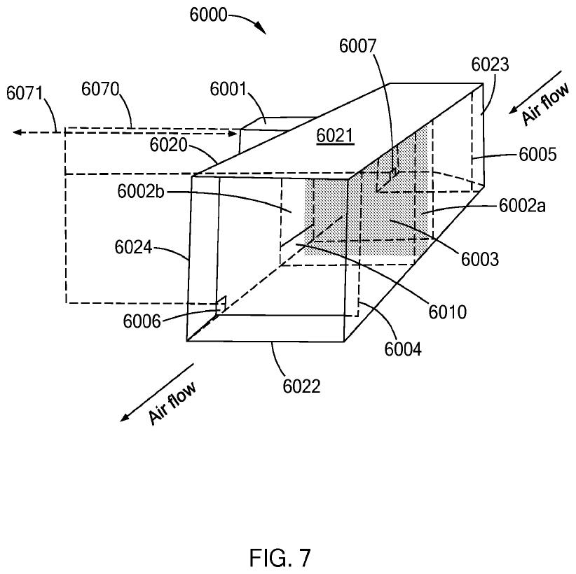

This air handling system 4000 has a blower (fan) 4001 for supply air, an airflow control assembly 4002a-c, dampers/flow regulator 4003a-c, supply air flow 4004, heating/cooling unit 4005, zones/rooms/areas 4010a-c, thermostats 4011a-c, airflow control assembly 4012, return air flow 4020, blower (fan) return air 4021, location(s) of one or more laser beam delivery units 4050 (e.g., blue laser diode assembly; e.g., FIG. 3 system; e.g. FIG. 7 system; e.g. FIG. 8 system)

### Example 4

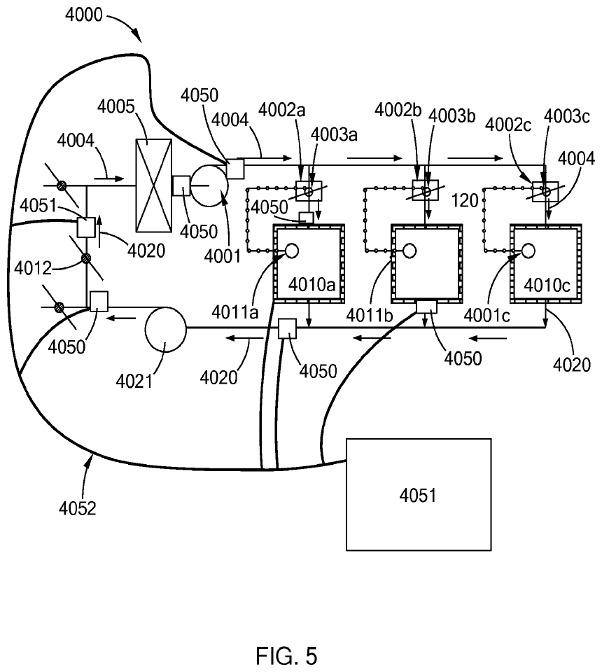

Turning to FIG. 5 there is shown a schematic of an embodiment of a laser air handling system having one or more laser delivery units. To the extent components of this system are the same as components of the system of FIG. 4 (Example 3) they have like numbers. The laser delivery units can be of the type of Example 4. Although not shown in the figure, a laser delivery unit can be positioned to processes incoming (fresh) air. In this embodiment each laser unit is connected by a high power optical fiber delivery system 4052 to transmit the laser beams from a laser 4051. The laser delivery units provide one or more sanitizing optical active zones.

### Example 5

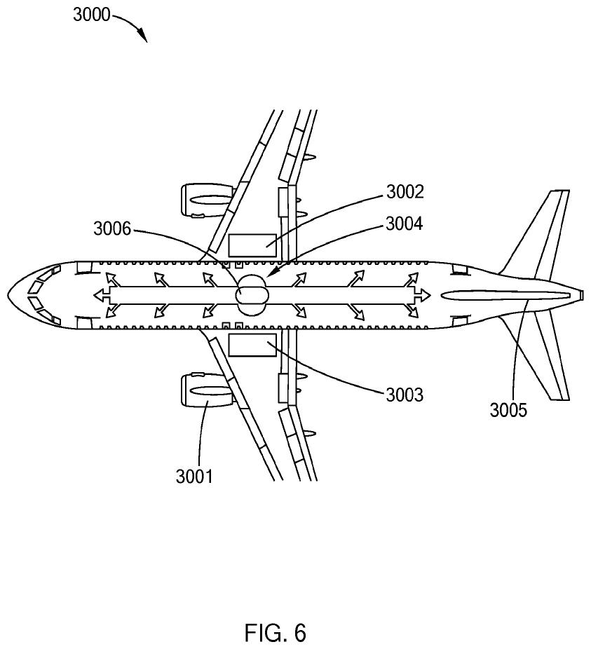

Turning to FIG. 6 there is shown a schematic of a laser sanitizing air handling system for an airplane. The laser delivery units provide one or more sanitizing optical active zones.

The airplane 3000 has an engine bleed 3001, a starboard air conditioning pack 3002, a port air condition pack 3003, an air handling, mixing and distribution system 3004, an APU (auxiliary power pack) and bleed 3005, and a Blue laser system for air processing 3006 (e.g., blue laser diode assembly; e.g., FIG. 3 system; e.g. FIG. 7 system; e.g. FIG. 8 system).

### Example 6

Turning to FIG. 7, there is shown a perspective view, with partial phantom lines showing internal structures as seen behind side panel 6023, a modular laser unit. One or more of these units can be installed in existing or new air handling systems by connecting the unit into the ducts of the system. The laser delivery units provide one or more sanitizing optical active zones.

The laser module 6000 (for insertion into, or use with, an HVAC system, e.g., to be connected into a duct) has a laser beam delivery assembly 6001 (e.g., laser source, diode laser source, optical fiber coupled to remote laser source), a first reflective optical surface (interior surface) 6002a, a second reflective optical surface (interior surface) 6002b, which faces the first optical surface 6002a, an optically active area defined by laser beam path in air flow 6003, a (up steam or incoming) filter/air permeable/optical blocking membrane 6004 to block laser beam (e.g., HEPA filter), a (downstream or outgoing) filter/air permeable/optical blocking membrane to block laser beam (e.g., HEPA filter) 6005, a sensor as part of safety interlock 6006, a sensor as part of safety interlock 6007, a residual beam management device 6010, a metal housing (e.g., air duct section) 6020, a wall of housing 6021, a wall of housing 6022, a wall of housing 6023, a wall of housing 6024, a safety interlock control communication 6070, and the system is in control communication 6071 with an HVAC control system.

### Example 7

Turning to FIG. 8 there is shown a schematic side view of an auxiliary or stand alone laser air sanitizing system 7000. The system can be added into or used with any existing air handling system to sanitize the air in that system. The laser delivery units provide one or more sanitizing optical active zones.

The system 7000 contains flow channels that are in fluid communication with an air inlet 7001 and an air outlet 7002. The flow channels can serially or in parallel channel/direct the incoming air from inlet 7001 through one or more sanitizing laser systems ((e.g., blue laser diode assembly; e.g., FIG. 3 system; e.g. FIG. 7 system) and then after the air flow has been sanitized to the outlet 7002.

### Example 8

The laser units in the embodiments of Examples 2 to 7 use the high power lasers and optical assemblies that are disclosed and taught in US Patent Publication Nos. 2021/0057865, 2020/0086388, 2016/0322777, 2018/0375296, 2016/0067827 and 2019/0273365, the entire disclosure of each of which is incorporated herein by reference.

### Example 9

The laser units in the embodiments of Examples 2 to 8 have laser beams where one or all of the beams has a wavelength in the range of 380 nm to 500 nm.

### Example 10

The laser units in the embodiments of Examples 2 to 8 have laser beams where one or all of the beams has a wavelength in the range of 405 nm to 495 nm.

### Example 11

The laser units in the embodiments of Examples 2 to 8 have laser beams where one or all of the beams has a wavelength in the range of 450 nm to 470 nm.

### Example 11A

The laser units in the embodiments of Examples 2 to 8 have laser beams where one or all of the beams has a wavelength the range of 700 nm to 1,500.

### Example 12

The laser units in the embodiments of Examples 2 to 8 have laser beams where one or all of the beams has a wavelength in the range of 500 nm to 575 nm.

### Example 13

The laser units in the embodiments of Examples 2 to 12 the laser units are Class I.

### Example 14

An HVAC system that is Class I and using an embodiment of Examples 2 to 12.

### Example 15

The delivery units and air handling systems of the embodiments of Examples 2-14 are used in, or for, any of the following places: theaters, airplanes, busses, airports, transportation stations, hotels, hospitals, medical facilities, churches, private homes, apartments, dormitories, mosques, temples, synagogues, office buildings, jails, automobiles, shopping malls, stores, arenas, schools, green houses, growing houses, poultry houses, chicken farms, horse barns, zoos and kennels.

### Example 16

A drone having a directed laser delivery unit, is autonomous flow in a pattern over an area to be sanitized by delivering the laser beam to that area.

### Example 17

A robot, a remotely operated vehicle, an autonomous vehicle, a preprogramed device is operated over an area to sanitize that area by delivering a sanitizing blue laser beam to the area. The laser beam delivery pattern is below a threshold where the contents of the area would be damaged.

### Example 18

The delivery units and air handling systems of the embodiments of Examples 2-15, which respect to the laser has a lifetime (and also can be accurately characterized, marketed and labeled, as having such lifetimes) of from about 5,000 hours to about 100,000 hours, from about 10,000 hours to about 90,000 hours, from about 5,000 hours to about 50,000 hours, from about 30,000 hours to about 70,000 hours, at least about 20,000 hours, at least about 30,000 hours, at least about 40,000 hours, at least about 50,000 hours and longer times.

### Example 19

The delivery units and air handling systems of the embodiments of Examples 2-15, which respect to the laser beams can have bandwidths of from about 10 pm (picometer) to about 10 nm, about 5 nm, about 10 nm, about 20 nm, from about 10 nm to about 30 nm, from about 5 nm to about 40 nm, about 20 nm or less, about 30 nm or less, about 15 nm or less, about 10 nm or less, as well as greater and smaller values.

It is noted that there is no requirement to provide or address the theory underlying the novel and groundbreaking performance or other beneficial features and properties that are the subject of, or associated with, embodiments of the present inventions. Nevertheless, various theories are provided in this specification to further advance the art in this important area, and in particular in the important area of lasers, laser processing and laser applications. These theories put forth in this specification, and unless expressly stated otherwise, in no way limit, restrict or narrow the scope of protection to be afforded the claimed inventions. These theories many not be required or practiced to utilize the present inventions. It is further understood that the present inventions may lead to new, and heretofore unknown theories to explain the operation, function and features of embodiments of the methods, articles, materials, devices and system of the present inventions; and such later developed theories shall not limit the scope of protection afforded the present inventions.

The various embodiments of lasers, laser devices, air handling systems, diodes, arrays, modules, assemblies, activities and operations set forth in this specification may be used in the above identified fields and in various other fields. Additionally, these embodiments, for example, may be used with: existing lasers, additive manufacturing systems, operations and activities as well as other existing equipment; future lasers, additive manufacturing systems operations and activities; and such items that may be modified, in-part, based on the teachings of this specification. Further, the various embodiments set forth in this specification may be used with each other in different and various combinations. Thus, for example, the configurations provided in the various embodiments of this specification may be used with each other. For example, the components of an embodiment having A, A′ and B and the components of an embodiment having A″, C and D can be used with each other in various combination, e.g., A, C, D, and A. A″ C and D, etc., in accordance with the teaching of this Specification. The scope of protection afforded the present inventions should not be limited to a particular embodiment, configuration or arrangement that is set forth in a particular embodiment, example, or in an embodiment in a particular Figure.

The invention may be embodied in other forms than those specifically disclosed herein without departing from its spirit or essential characteristics. The described embodiments are to be considered in all respects only as illustrative and not restrictive.

1. A laser system having a sanitizing laser beam to mitigating harmful materials in a gas stream, the laser system comprising:
a. a laser for generating a sanitizing laser beam along a laser beam path;
b. a housing, the housing defining an optically active area;
c. wherein, the optically active area is on the laser beam path and thereby in optical communication with the laser;
d. the laser beam path extended into the optically active area; and thereby defining a portion of the laser beam path as an optically active laser beam path; whereby the optically active laser beam path is located within the optically active area; and,
e. the optically active area configured to have a gas stream flow through the optically active area;
f. whereby during operation the gas stream flows through the sanitizing laser beam on the optically active laser beam path.

2. The laser system of claim 1, comprising optics for defining the shape of the laser beam.

3. The laser system of claim 2, wherein the laser beam in the optically active area comprises a beam cross section shape selected from the group consisting of circular, square, rectangular, and linear.

4. The laser system of claim 1, wherein a plurality of beam directing devices are located on the optically active laser beam path.

5. The laser systems of claim 4, wherein the plurality of beam directing devices comprise a first reflecting device and a second reflecting device, wherein each reflecting device is oriented at an angle to the laser beam path, wherein the angle is other than 90°.

6. The laser systems of claim 5, wherein the angle is from about 0.5° to about 15°.

7. (canceled)

8. (canceled)

9. (canceled)

10. The laser systems of claim 4, wherein one, both or all of the reflecting devices comprises a reflecting device selected from the group consisting of a mirror, a mirror having at least 99% reflectivity for the laser, and a total internal reflection optic.

11-32. (canceled)

33. A laser system having a sanitizing laser beam to mitigating harmful materials in a gas stream, the laser system comprising:
a. a laser for generating a laser beam along a laser beam path;
b. an optically active area, comprising a plurality of laser beam directing devices to define an optically active laser beam path; the optically active laser beam path defining an illumination zone; and,
c. the optically active laser beam path in optical communication with the laser beam path, and thereby forming a part of the laser beam path;
d. wherein, the system is configured to provide a laser power density in the illumination zone to mitigate a harmful material.

34. The laser system of claim 33, comprising optics for defining the shape of the laser beam.

35. The laser system of claim 34, wherein the laser beam in the optically active area comprises a beam cross section shape selected from the group consisting of circular, square, rectangular, and linear.

36. The laser systems of claim 33, wherein a plurality of beam directing devices are located on the optically active laser beam path.

37. The laser systems of claim 36, wherein the plurality of beam directing devices comprise a first reflecting device and a second reflecting device, wherein each reflecting device is oriented at an angle to the laser beam path, wherein the angle is other than 90°.

38. The laser systems of claim 37, wherein the angle is from about 0.5° to about 15°.

39-64. (canceled)

65. A laser system having a sanitizing laser illumination zone for mitigating harmful materials, the laser system comprising:
a. a laser for generating a laser beam along a laser beam path;
b. an optically active area, defining an illumination zone;
c. wherein, at least a portion of the laser beam path is within the optically active area; and,
d. the laser beam path extended into the optically active area; and thereby defining a portion of the laser beam path as an optically active laser beam path; whereby the optically active laser beam path is located within the optically active area; and,
e. the system is configured whereby the illumination zone is a sanitizing illumination zone.

66. The laser system of claim 65, comprising optics for defining the shape of the laser beam.

67. The laser system of claim 66, wherein the laser beam in the optically active area comprises a beam cross section shape selected from the group consisting of circular, square, rectangular, and linear.

68. The laser systems of any of the foregoing claims, wherein a plurality of beam directing devices are located on the optically active laser beam path.

69. The laser systems of claim 68, wherein the plurality of beam directing devices comprise a first reflecting device and a second reflecting device, wherein each reflecting device is oriented at an angle to the laser beam path, wherein the angle is other than 90°.

70. The laser systems of claim 69, wherein the angle is from about 0.5° to about 15°.

71-123. (canceled)
